(19) United States
(12) Patent Application Publication
(10) Pub. No.: US 2012/0132212 A1
(43) Pub. Date: May 31, 2012

(54) Endotracheal Tube with Multi-Mode Valve and Method of Using Same

(75) Inventor: Vasu Nishtala, Snellville, GA (US)

(73) Assignee: C. R. BARD, INC., Murray Hill, NJ (US)

(21) Appl. No.: 13/260,663

(22) PCT Filed: Mar. 23, 2010
(86) PCT No.: PCT/US2010/028342

§ 371 Date: Dec. 2, 2011

Related U.S. Application Data
(60) Provisional application No. 61/163,956, filed on Mar. 27, 2009.

(51) Int. Cl. A61M 16/04

(52) U.S. Cl. 128/207.15

(57) ABSTRACT

An endotracheal tube for intubation within a patient's trachea. The endotracheal tube includes an elongated tube having a main lumen, an auxiliary lumen, a proximal end, and a distal end. An inflatable cuff is arranged on the elongated tube between the proximal and distal ends. A multi-mode valve is arranged on the elongated tube. A method for tracheal intubation of a patient is also disclosed.

### CROSS REFERENCE TO RELATED APPLICATIONS

The instant application is a US National Stage of International Application No. PCT/US2010/028342 filed on Mar. 23, 2010 and published as WO 2010/111287 on Sep. 30, 2010, which is based on U.S. provisional application No. 61/163,956, filed Mar. 27, 2009, the disclosure of which is hereby expressly incorporated by reference hereto in its entirety.

### STATEMENT CONCERNING GOVERNMENT INTEREST

Not applicable.

### BACKGROUND OF THE INVENTION

### Field of the Invention

The invention relates to an endotracheal (ET) tube utilizing an elongated tube having a main lumen, an auxiliary lumen, a proximal end, and a distal end. An inflatable cuff is arranged on said elongated tube between said proximal and distal ends. A multi-mode valve is arranged on the elongated tube

### BACKGROUND OF THE INVENTION

Ventilator-associated pneumonia can be a cause of morbidity in critically ill patients. Approximately 250,000 cases of Ventilator Associated Pneumonia (VAP) are reported each year. The mortality associated with VAP is approximately 23,000 patients annually. (Engelmann, J. et al.; Ventilator-Associated Pneumonia, Seminars in Infection Control, Vol. 1, No. 2, 2001).

VAP may be associated with the long-term use of invasive positive pressure medical devices such as mechanical ventilators and tracheal tubes, as well as suction catheters, gastric feeding tubes, esophageal obturators, esophageal balloon catheters, bronchoscopes, breathing circuits, filters, heat and moisture exchanges, and humidifiers. Tracheal tubes include airway management devices such as endotracheal tubes, tracheostomy tubes, and transtracheal tubes.

Tracheal intubation is used in respiratory medicine to deliver or remove a fluid to the airways of a patient. Tracheal intubation with an endotracheal tube is commonly used during general anesthesia and when critically ill patients require airway protection and mechanical ventilation. Under certain conditions of long term ventilation, a tracheostomy tube is inserted through a surgical opening through the neck. An endotracheal tube is inserted into the trachea through either the mouth or nose (nasotracheal tube). Tracheal tubes are used for ventilation and removal of secretions. Typically, endotracheal tubes are disposable plastic tubes, easily placed through the mouth or nose, that guarantee a patent conduit for the delivery of respiratory gases. A seal between the outer wall of the tracheal tube and the inner lining of the trachea (the tracheal mucosa) must typically be formed. Most endotracheal tubes provide a very compliant, thin walled inflatable cuff that forms a seal with the proximal tracheal rings. This seal allows for positive pressure ventilation at normal airway pressures with minimal leakage. The seal thus provides a closed circuit for ventilation and is configured to minimize or prevent aspiration of pharyngeal contents into the respiratory tract. This seal is usually formed by inflating the pressurized cuff that surrounds the tracheal tube.

Bacteria may flow to or accumulate in the area immediately before or adjacent the cuff and pool there, eventually becoming sessile on the outer surface of the endotracheal tube. Microorganisms may also adhere to abiotic surfaces and allow complex biofilms to form. The complex biofilm may protect the microorganisms against antibiotic action. The accretion of antibiotic-resistant biofilms may form a reservoir of infecting microorganisms which may then migrate from the endotracheal outer surface past the protective cuff and contaminate the trachea and lungs. Lung secretions containing microorganisms, blood, mucous, and cellular debris may, for example, colonize on the tip and inner lumen of the endotracheal tube to form biofilms of antibiotic-resistant microorganisms. Such interluminal biofilms may occlude the breathing tube or migrate back into the lungs to cause further infection.

The process of removing these biofilms and secretions with conventional suction catheters may lead to the aspiration of fragments of biofilms or infected aerosols. Contaminated suction catheters, feeding tubes, ventilator tubing and breathing circuits, filters, heat and moisture exchangers, nebulizers, heated humidifiers, and other related breathing tubes or devices may be sources of microorganism contamination and thus may contribute to biofilm formation.

One method of mitigating colonization of tube surfaces by bacteria is by suctioning. Routine suctioning of sub-glottic secretions may be associated with significant reduction of VAP. The drainage of sub-glottic secretions can be performed using an endotracheal tube with a suction lumen, such as a Mallinkrodt HI-LO EVAC endotracheal tube. This tube has an elliptic dorsal opening above the cuff that is connected to a separate, integral aspiration lumen. A small bore suction channel is incorporated into the wall of the tube with its distal opening above (and distal to) the inflated cuff. It is noted, however, that prior study of the instant type of endotracheal tube by this inventor's laboratory found a high incidence of suction channel blockage with long term use. In addition, the rate and viscosity of removed secretions is greatly limited by the small lumen.

Therefore, in a long term intubation subject, bacteria can multiply in the pool of secretions that accumulate above the inflated cuff, providing a significant inoculum when aspirated into the distal trachea. Irrigation and drainage are the methods of choice when attempting to remove infected material from the body. Irrigation dilutes the bacterial colony count and suction drainage removes this material from the body. Applying this technique to the area below the vocal cords and above the inflated cuff (sub-glottic region), may reduce the incidence and amount of infected secretions that reach the lungs and therefore prevent nosocomial pneumonias. Sub-glottic irrigation with a bactericidal solution may also significantly decrease the infectivity of secretions that may be aspirated into the distal trachea. The following published documents (each of which is incorporated by reference herein in its entirety) are representative of the state of the art: U.S. Patent Application Publications 2006/0118121, 2006/0107962, and 2004/0220534; U.S. Pat. Nos. 5,819,723 and 5,582,167; as well as WO 02/076279.

Despite several successes in the field of endotracheal tubes, the afore-mentioned documents have fallen short of addressing the fact that, since the endotracheal tube in-dwells in the trachea, the suction opening has a tendency to rub against tissue and inflame the localized area. In addition, although the opening is located on the posterior of the endotracheal tube, due to its small size, it has limited reach.

### SUMMARY OF THE INVENTION

The invention relates to embodiments that overcome one or more of the deficiencies or disadvantageous noted above.

The invention also provides for an endotracheal (ET) tube comprising an elongated tube having a main lumen, an auxiliary lumen, a proximal end, and a distal end. An inflatable cuff is arranged on said elongated tube between said proximal and distal ends. A multi-mode valve is arranged on the elongated tube.

The ET tube may be configured for intubation and/or positionable inside a trachea. The proximal end may be configured to extend outside a trachea. The elongated tube may have a shape configured to substantially conform to an anatomical cross-section of a human trachea. The auxiliary lumen may extend longitudinally along a length of said elongated tube and is structured and arranged to remove secretions from a trachea. The auxiliary lumen may be coupled to an auxiliary chamber formed in a wall of the elongated tube.

The multi-mode valve may be coupled to and/or in fluid communication with the auxiliary lumen. The multi-mode valve may be coupled to the auxiliary lumen and the auxiliary lumen is a suction lumen. The multi-mode valve may be coupled to the auxiliary lumen and the auxiliary lumen is coupled to at least one of a suction source and a pressure source. The multi-mode valve may be coupled to the auxiliary chamber and the auxiliary lumen is coupled to at least one of a suction source and a pressure source. The valve may also be a fluid-flow regulating device, a passive fluid-flow regulating device, and/or a pressure responsive fluid-flow regulating device

At least a portion of said elongated tube may have a cross-sectional shape that is one of substantially round, substantially oval, substantially egg-shaped, and substantially triangular.

The cuff may have a cross-sectional shape that is one of substantially round, substantially oval, substantially egg-shaped, and substantially triangular.

The multi-mode valve may comprise at least the following modes of operation; normally sealed or closed mode and at least one open mode allowing fluid and/or gas to pass into and/or out of the elongated tube.

The multi-mode valve may comprise the following modes of operation; normally sealed or closed mode, a first open mode allowing fluid and/or gas to pass into the elongated tube, and a second open mode allowing fluid and/or gas to pass out of the elongated tube.

The multi-mode valve may comprise the following modes of operation; a normally sealed or closed mode, a first open mode allowing fluid and/or gas to pass into the auxiliary lumen, and a second open mode allowing fluid and/or gas to exit out of the multi-mode valve from the auxiliary lumen.

The multi-mode valve may be a three-way valve. The multi-mode valve may comprise at least one of a slit valve, at least two deflectable edges arranged adjacent one another, a valve positioned arranged an outer wall of the elongated tube and being substantially flush with an outer surface of the outer wall of the elongated tube, and a valve integrally formed in an outer wall of the elongated tube and being substantially flush with an outer surface of the outer wall of the elongated tube. The multi-mode valve may be substantially flush with an outer surface of an outer wall of said elongated tube.

The multi-mode valve may comprise a slit extending through an outer surface of an outer wall of said elongated tube, said slit being substantially parallel to and offset from a longitudinal axis of said elongated tube and being defined by opposed, aligned, normally sealed, parallel first and second edges of respective oppositely disposed first and second wall segments. At least one of said first and second wall segments may be pivotably arranged or deflectable from an original position in response to a predetermined pressure differential between a pressure in said auxiliary lumen and a pressure exterior to said elongated tube, whereby at least one of said first and second wall segments is capable of selectively opening either outwardly and/or inwardly.

The multi-mode valve may be structured and arranged to permit traumatic tamponade against a trachea.

The ET tube may further comprise a ring located at said distal end of said elongated tube. The ET tube may further comprise a suction port coupled to the auxiliary lumen and an inflation port coupled to the cuff. The ET tube may further comprise a suction port coupled to the auxiliary lumen and an another lumen arranged inside the elongated tube configured to introduce fluid to an area outside the elongated tube.

The elongated tube may be made of a polymeric material. The cuff may be located at least one of below said multi-mode valve and closer to the distal end than the multi-mode valve.

The auxiliary lumen may be structured and arranged to introduce a fluid into a trachea. The ET tube may further comprise a second lumen arranged inside the elongate tube for at least one of removal of secretions from an area of a trachea and introducing fluid into an area of the trachea.

The invention also provides for a method for tracheal intubation comprising inserting the ET tube described above into a trachea and inflating the inflatable cuff to enable positive pressure ventilation.

The method may utilize the following modes of the multi-mode valve; a normally sealed or closed mode, a first open mode allowing fluid and/or gas to pass into the auxiliary lumen, and a second open mode allowing fluid and/or gas to exit out of the multi-mode valve from the auxiliary lumen.

The invention also provides for an ET tube comprising an elongated tube having a main lumen, an auxiliary chamber, a proximal end, and a distal end. An inflatable cuff is arranged on said elongated tube between said proximal and distal ends. A multi-mode valve is arranged on the elongated tube and in fluid communication with the auxiliary chamber. The multi-mode valve comprises the following modes of operation; a normally sealed or closed mode, a first open mode allowing fluid and/or gas to pass into the elongated tube, and a second open mode allowing fluid and/or gas to pass out of the elongated tube.

The invention also provides for an ET tube comprising an elongated tube having a main lumen, an auxiliary chamber, a proximal end, and a distal end. An inflatable cuff is arranged on said elongated tube between said proximal and distal ends. A multi-mode valve is integrally formed with an outer wall of the elongated tube and in fluid communication with the auxiliary chamber. The multi-mode valve comprises the following modes of operation; a normally sealed or closed mode, a first open mode allowing fluid and/or gas to pass into the auxiliary chamber based on a first pressure difference between opposite sides of the multi-mode valve, and a second open mode allowing fluid and/or gas to pass out of the auxiliary chamber based on a second pressure difference between opposite sides of the multi-mode valve.

In other embodiments, there is provided an endotracheal tube for intubation within a patient's trachea. The endotracheal tube includes an elongated tube having a main chamber, an auxiliary chamber, a proximal end and a distal end, the distal end for insertion within the patient's trachea and the proximal end for positioning outside the patient's trachea, the elongated tube having a shaped cross-section for substantially conforming to the anatomical cross-section of the patient's trachea, a first lumen longitudinally extending along a length of the elongated tube for removal of secretions from the trachea, the lumen having a proximal end and a distal end and an inflatable cuff positioned on the elongated tube between the proximal and distal ends.

In embodiments, the endotracheal tube includes a three-way valve, the three-way valve positioned within an outer wall of the elongated tube and substantially flush with an outer surface of the outer wall of the elongated tube and proximal to the distal end of the tube, the three-way valve in fluid communication with the auxiliary chamber.

In embodiments, the three-way valve includes a normally sealed, selectively operable, laterally disposed, three-position slit valve communicating between the auxiliary chamber and the outer wall of the elongated tube, the slit valve affording selective influent and effluent flow control into and out of the auxiliary chamber.

In embodiments, the three-way valve comprises a slit extending through the outer surface of the outer wall of the elongated tube, the slit being substantially parallel to and offset from a longitudinal axis of the elongated tube and being defined by opposed, aligned, normally sealed, parallel first and second edges of respective oppositely disposed first and second slit valve wall segments.

In still other embodiments, the first slit valve wall segment is pivotably displaceable inwardly and outwardly responsive to predetermined pressure differentials between the pressure in the auxiliary chamber and the pressure exterior to the elongated tube, thereby to pivot the first edge into a spaced-apart spatial relationship from the second edge, whereby the first edge is capable of selectively opening either outwardly from a normally sealed position to infuse fluid from the auxiliary chamber into a space exterior to the distal end of the elongated tube, or inwardly from the normally sealed position to withdraw fluid from the space exterior to the distal end of the elongated tube into the auxiliary chamber, the first slit valve wall segment comprises a relatively thin localized site in the material from which the first slit valve wall segment is comprised.

In embodiments, the three-way valve permits tamponade against the trachea.

In embodiments, a suction port is located at the distal end of the first lumen.

In embodiments, the elongated tube is made of a polymeric material.

In embodiments, a second lumen extends along the inner wall of the main chamber for removing secretions from the trachea or introducing fluid into the trachea.

In embodiments, there is provided a method for tracheal intubation of a patient, comprising the steps of inserting an endotracheal tube into the patient's trachea, the endotracheal tube comprising (i) an elongated tube having a main chamber and an auxiliary chamber, and a proximal end and a distal end, the distal end for insertion within the patient's trachea and the proximal end for positioning outside the patient's trachea, the elongated tube having a shaped cross-section for substantially conforming to the anatomical cross-section of the trachea; (ii) a first lumen longitudinally extending along a length of the elongated tube for removal of secretions from the trachea, the lumen having a proximal end and a distal end; and (iii) an inflatable cuff positioned on the elongated tube between the proximal and distal ends and inflating the inflatable cuff to enable positive pressure ventilation.

These and other features will be apparent from the detailed description taken with reference to accompanying drawings.

### BRIEF DESCRIPTION OF THE DRAWINGS

Further explanation is provided in the description that follows with reference to the drawings illustrating, by way of non-limiting examples, various forms, wherein:

FIG. 1 shows a side perspective view of a sub-glottic endotracheal tube according to one form disclosed herein;

FIG. 2 shows another side view of the sub-glottic endotracheal tube of FIG. 1;

FIG. 3 shows an enlarged view of a section of the sub-glottic endotracheal tube of FIG. 1, illustrating a three-way valve according to one form disclosed herein;

FIG. 4 shows a perspective cross-sectional view of the sub-glottic endotracheal tube, taken along line 4-4 of FIG. 1;

FIG. 5 shows a perspective cross-sectional view of the sub-glottic endotracheal tube, taken along line 5-5 of FIG. 2; and

FIG. 6 shows a cross-sectional view of a patient's trachea.

### DETAILED DESCRIPTION OF THE INVENTION

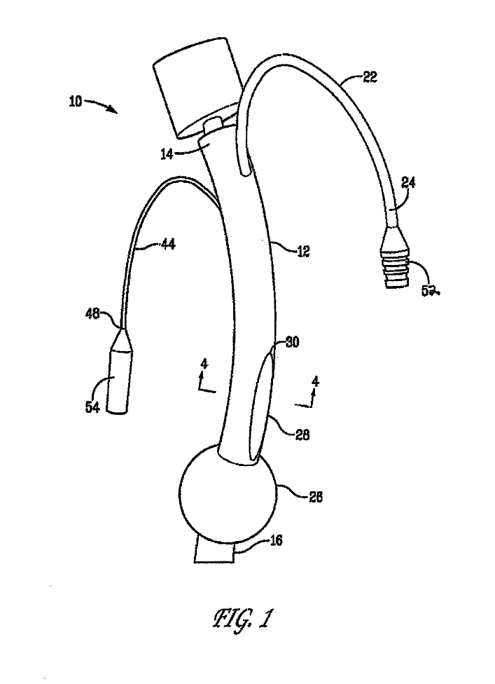

Various aspects will now be described with reference to specific forms selected for purposes of illustration. It will be appreciated that the spirit and scope of the apparatus and methods disclosed herein are not limited to the selected forms. Moreover, it is to be noted that the figures provided herein are not drawn to any particular proportion or scale, and that many variations can be made to the illustrated forms. Reference is now made to FIGS. 1-6, wherein like numerals are used to designate like elements throughout.

The term “distal end” refers to the end of the catheter which is inserted into the patient's body.

The term “proximal end” refers to the end thereof which is situated externally of a patient's body.

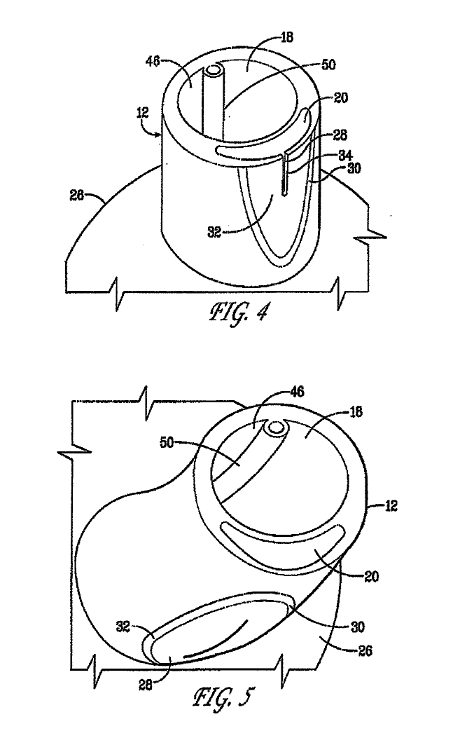

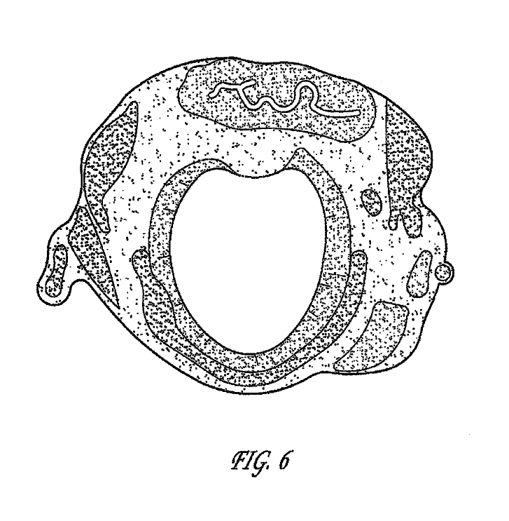

Referring now to FIGS. 1-5, an endotracheal tube 10 for intubation within a patient's trachea is shown. The endotracheal tube 10 includes an elongated tube 12 having a proximal end 14 and a distal end 16. Distal end 16 is for insertion within the patient's trachea and proximal end 14 is for positioning outside the patient's trachea. As may be seen by reference to FIGS. 4-5, elongated tube 12 may be provided with a substantially oval-shaped cross-section for substantially and/or generally conforming to the anatomical cross-section of the patient's trachea (see FIG. 6). According to one embodiment, the elongated tube 12 has a substantially egg-shaped cross section. According to another embodiment, the elongated tube has a substantially oval cross sectional shape, or a substantially round cross sectional shape. According to yet another embodiment, the cross sectional shape is substantially triangular. The elongated tube 12 may also have a varying shape along its length. A cuff 26 is arranged on the tube 12. According to certain embodiments, the cuff 26 may have a cross-sectional shape that is substantially round, oval, egg-shaped, or triangular. The tube 12 also includes an outer wall 30.

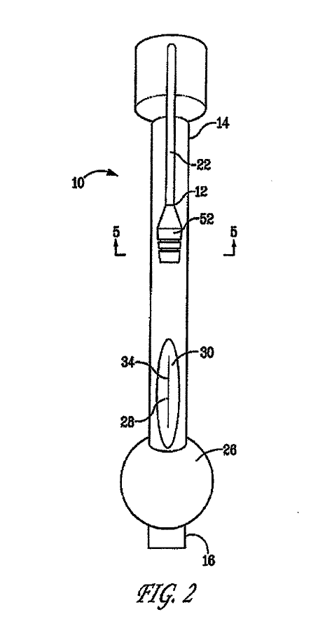

Referring now to FIGS. 4 and 5, elongated tube 12 of endotracheal tube 10 includes a main chamber 18 and an auxiliary chamber 20. As shown in FIGS. 1 and 2, a first lumen or cunduit 22 is provided that longitudinally extends within auxiliary chamber 20 of elongated tube 12 for the removal of secretions from the trachea. According to one embodiment, the first lumen 22 has a proximal end 24 and a distal end (not shown) in fluid communication with auxiliary chamber 20. A suction port or connector 52 may be provided at the proximal end of first lumen 22.

The inflatable cuff 26 is positioned on elongated tube 12 between proximal end 14 and distal end 16. In embodiments, the inflatable cuff 26 is arranged on the tube 12 closer to the distal end 16 than the proximal end 14, and forms a seal with a trachea around elongated tube 12 to block secretions that may otherwise be aspirated. This seal can allow for positive pressure ventilation at normal airway pressures with minimal leakage. The seal thus provides a closed circuit for ventilation and also prevents aspiration of pharyngeal content into the respiratory tract. In one form, inflatable cuff 26 is formed of a material that swells in thickness upon absorbing moisture from the surrounding tissues providing an ability to seal at a lower pressure, such as a lower contact pressure between inflatable cuff 26 and the surrounding tissues or the trachea.

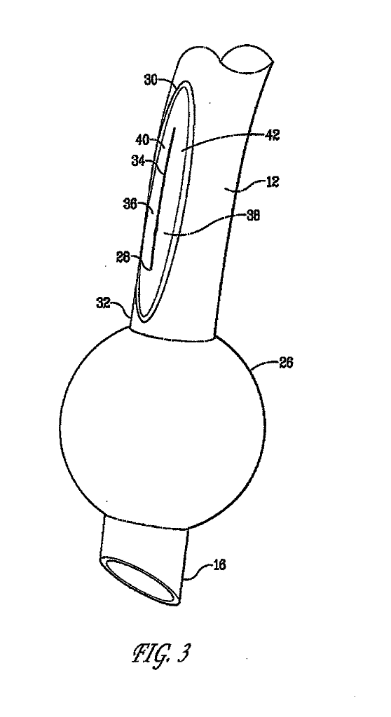

Endotracheal tube 10 also includes a three-way valve 28 positioned within an outer wall 30 of elongated tube 12. As shown in detail in FIG. 3, in embodiments, the three-way valve 28 is substantially flush with an outer surface 32 of outer wall 30 of elongated tube 12 and proximal to the distal end 16 of elongated tube 12. As shown in FIGS. 4 and 5, three-way valve 28 is in fluid communication with auxiliary chamber 20.

In embodiments, the three-way valve 28 is a normally sealed, selectively operable, laterally disposed, three-position slit valve that communicates between auxiliary chamber 20 and outer wall 30 of elongated tube 12. Such a slit valve affords selective influent and effluent flow control into and out of the auxiliary chamber 20. This type of a valve is known as a Groshong valve and is disclosed in, e.g., U.S. Pat. No. 4,549,879 (the disclosure of which is hereby expressly incorporated by reference in its entirety). In embodiments, the Groshong valve may be designed to remain closed between about −7 and about 80 mm Hg. Groshong-valved catheters are available from Bard Access Systems (Salt Lake City, Utah).

Referring to FIG. 3, the three-way valve. 28 includes a slit 34 extending through outer surface 32 of outer wall 30 of elongated tube 12. As shown, slit 34 may be substantially parallel to and offset from a longitudinal axis of elongated tube 12. Slit 34 is defined by opposed, aligned, normally sealed, parallel first and second edges 36 and 38 of respective oppositely disposed first and second slit valve wall segments 40 and 42. According to one embodiment, the distance between the proximal end of slit 34 and cuff 26, ranges from about 0.5 to about 15 mm, for example about 2 to about 10 mm, such as about 5 mm. According to another embodiment, the valve 28 (and thus slit 34) may be disposed horizontally on outer wall 32, whereby the slit 34 extends along a direction normal (i.e., perpendicular) to the longitudinal axis of the elongated tube 12.

The three-way valve 28, in embodiments, may have the following three modes of operation; a first open position wherein one or both of slit edge segments 36 and 38 deflect inwardly into the auxiliary chamber 20. In embodiments, this can occur due to negative and/or suction pressure in the chamber 20. This allows fluids and/or gasses outside the valve 28 to be suctioned into the chamber 20. A second open position of the valve 28 occurs when one or both of slit edge segments 36 and 38 deflect outwardly. In embodiments, this can occur due to positive and/or increased pressure in the chamber 20. This allows fluids and/or gasses inside the chamber 20 to exit out of the valve 28. A third or closed position of the valve 28 occurs when both of slit edge segments 36 and 38 are in a relaxed or original position such that the edges 36 and 38 essentially contact each other and/or sealingly engage each other. In embodiments, this can occur when the pressure inside the chamber 20 is essentially the same as the pressure outside the tube 12. This essentially prevents fluids and/or gasses inside the chamber 20 from exiting out of the valve 28 and vice versa.

In embodiments, the first slit valve wall segment 40 is pivotably displaceable inwardly and outwardly responsive to predetermined pressure differentials between the pressure in auxiliary chamber 20 and the pressure exterior to elongated tube 12. Thus, the first edge 36 can pivot into a spaced-apart spatial relationship from or relative to the second edge 38. The first edge 36 is thus capable of selectively opening either outwardly from a normally sealed position to infuse fluid from auxiliary chamber 20 into a space exterior to distal end 16 of elongated tube 12, or inwardly from the normally sealed position to withdraw fluid from the space exterior to distal end 16 of elongated tube 12 into auxiliary chamber 20. In embodiments, the first slit valve wall segment 40 may be formed from a relatively thin localized site in the material from which first slit valve wall segment 40 is comprised. In another form, second slit valve wall segment 42 may be formed from a relatively thin localized site in the material from which second slit valve wall segment 42 is comprised.

Referring to FIGS. 1, 4 and 5, a second lumen 44 may be provided that extends along inner wall 46 of main chamber 18 for removal of secretions from the trachea or introducing fluid into the trachea. Second lumen 44 has a proximal end 48 and port or connector 54, and a distal end 50. A third lumen (not shown) may be provided that extends along inner wall 46 of main chamber 18 and is in fluid communication with inflatable cuff 26 for inflating inflatable cuff 26.

In embodiments, there is provided a method for tracheal intubation of a patient, comprising the steps of inserting an endotracheal tube 10 into the patient's trachea, wherein the endotracheal tube 10 comprises (i) an elongated tube 12 having a main chamber 18 and an auxiliary chamber 20, and a proximal end 14 and a distal end 16, the distal end 16 for insertion within the patient's trachea and the proximal end 14 for positioning outside the patient's trachea, the elongated tube 12 having an cross-section configured to substantially conform to the anatomical cross-section of the trachea; (ii) a first lumen 22 longitudinally extending within the auxiliary chamber 20 of the elongated tube 12 for removal of secretions from the trachea, the lumen having a proximal end 24 and a distal end (not shown) and (iii) an inflatable cuff 26 positioned on elongated tube 12 between the proximal and distal ends 14 and 16; and inflating the inflatable cuff 26 to enable positive pressure ventilation.

As may be appreciated, in ventilated patients using an endotracheal tube, bacteria may flow to the area immediately before the cuff and pool there, eventually becoming sessile on the outer surface of the endotracheal tube. Microorganisms may adhere to an abiotic surface and allow complex biofilms to form. Particles of biofilm may fall into the lungs during intubation. These particles may produce VAP. VAP may also be produced by simple planktonic (free-floating) single cell microbes that may come from leakage around the cuff, or air entering from the lumen of an endotracheal tube. The complex biofilm may protect the microorganisms against antibiotic action. Preventing the downward leak of the secretions is can be important to preventing VAP.

Because an endotracheal tube in-dwells in the trachea, the suction holes of conventional devices have a tendency to rub against tissue and inflame the localized area. Friction between an endotracheal tube and surrounding tissue may cause irritation in the surrounding tissues such as vocal cords. Such friction may make it more difficult to insert an endotracheal tube in the patient's trachea and may also produce trauma to the surrounding tissues. The endotracheal tubes disclosed herein are formed of a soft and planar material, so as to have a lower surface friction and address this issue. This is particularly the case with the three-way valve 28 disclosed herein, as may be appreciated. In a further form, the three-way valve permits atraumatic tamponade against the trachea. In the event the suction hole or slit rubs against the surrounding tissue, the material (which in embodiments may be made of silicone) serves to reduce friction between the endotracheal tube and surrounding tissue, thereby reducing irritation in the vocal cords. In addition, the inherent quality of silicone allows the shape of the silicone suction valve to take several additional forms to provide a greater reach for the removal of secretions from the trachea.

Elongated tube 12 may be formed from a polymeric material. In one form, the polymeric material is a hydrophobic material such as polyvinylchloride (PVC). In one form, elongated tube 12 may be partially translucent so that the physician can determine the presence of fluid therein. To inhibit microbes from attaching themselves to a hydrophobic surface, a hydrophilic layer may be applied over the hydrophobic surface. The hydrophilic coating may be, e.g., a polyurethane such as medical grade hydrophilic thermoplastic polyurethane. Hydrophilic coatings may be applied by a coating operation, as those skilled in the art readily understand.

According to various embodiments, at least a portion of the endotracheal tube disclosed herein may be coated with at least one bioactive composition, such as an antimicrobial composition. For example, the endotracheal tube can comprise an antimicrobial coating, such as one of those described in U.S. Pat. No. 6,716,895 (the entire disclosure of which is incorporated herein by reference).

As may be appreciated, aspects of the enhanced sub-glottic suction endotracheal tube disclosed herein may apply in various forms to many types of medical devices, such as tubes, catheters, stents, feeding tubes, breathing circuits, intravenous tubes, breathing tubes, circuits, and related airway accessories such as connectors, adapters, filters, humidifiers, nebulizers, and prosthetics as well.

As noted hereinabove, the enhanced sub-glottic endotracheal tubes disclosed herein can have, by way of non-limiting example, a substantially oval shaped cross-section, a substantially egg-shaped cross-section, a substantially round cross-section, or a substantially triangular cross-section. The selected shape of the tube may allow it to substantially conform to the anatomical cross section of the trachea, as shown in FIG. 6. The endotracheal tubes disclosed herein reduce friction between the endotracheal tube and surrounding tissues and reduce irritation in the vocal cords. In addition, the shape of the tube allows for incorporation of one or more lumens extending along the inner wall of the oval shaped tube's main chamber, between the proximal and distal ends of the tube. The secretions would be evacuated into this lumen and out of the patient's trachea. In a further form, a ring is located at the most distal end of the oval shaped tube.

In one form, it may be desirable to determine the carbon dioxide content of respiration, so as to determine whether the intubation is proper. It would further be desirable if a hydrophilic surface of the endotracheal tube were injected with a chemical, such as an acid-base color dye, to indicate CO₂ concentration.

As indicated hereinabove, three-way valve 28 may be formed of a durable, flexible, biocompatible material such as silicone rubber. Typically, such material may have a hardness of less than 100 durometer and an elongation percentage of up to 700. In one form, the material is a silicone rubber tubing having a hardness of about 59 durometer, sold under the trademark SILASTIC by Dow Corning Co. of Midland, Mich.

In one form, it may be desirable that the three-way valve 28 and the valve faces are treated with a biocompatible chemical which will focally weaken and make the material more pliable so that the treated portion is more easily deformable upon the application of pressure gradients across the three-way valve 28, thus facilitating valve function. One suitable agent is a dimethylsiloxane containing material sold by Dow Corning Co. under the designation Dow Corning Fluid 360. By applying said chemical to the valve surfaces as well as the surrounding internal and external walls for at least one minute, sufficient dimethylsiloxane is adsorbed into the silicon three-way valve 28 to weaken the wall contiguous the valve for an extended period of time. After treatment with the chemical the valve will open to allow outward flow of fluid at a lesser pressure gradient than if the valve had not been treated. Likewise, a lesser negative or suction pressure is necessary to cause fluid to be withdrawn into the catheter. Thus, the essential valve function of the three-way valve 28 is facilitated.

When numerical lower limits and numerical upper limits are listed herein, any value (whole number or otherwise) within the ranges from any lower limit to any upper limit are contemplated.

All patents, test procedures, and other documents cited herein, including priority documents, are fully incorporated by reference to the extent such disclosure is not inconsistent and for all jurisdictions in which such incorporation is permitted.

While the illustrative forms disclosed herein have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside herein, including all features which would be treated as equivalents thereof by those skilled in the art to which this disclosure pertains.

1. An endotracheal (ET) tube, comprising:
an elongated tube having a main lumen, an auxiliary lumen, a proximal end, and a distal end;
an inflatable cuff arranged on said elongated tube between said proximal and distal ends; and
a multi-mode valve arranged on the elongated tube.

2. (canceled)

3. The ET tube of claim 1, wherein the proximal end is configured to extend outside a trachea.

4. The ET tube of claim 1, wherein the elongated tube has a shape configured to substantially conform to an anatomical cross-section of a human trachea.

5. The ET tube of claim 1, wherein the auxiliary lumen extends longitudinally along a length of said elongated tube and is structured and arranged to remove secretions from a trachea.

6. The ET tube of claim 1, wherein the multi-mode valve is coupled to and/or in fluid communication with the auxiliary lumen.

7. The ET tube of claim 1, wherein the multi-mode valve is coupled to the auxiliary lumen and the auxiliary lumen is a suction lumen.

8. The ET tube of claim 1, wherein the multi-mode valve is coupled to the auxiliary lumen and the auxiliary lumen is coupled to at least one of:
a suction source; and
a pressure source.

9. The ET tube of claim 1, wherein the auxiliary lumen is coupled to an auxiliary chamber formed in a wall of the elongated tube.

10. The ET tube of claim 9, wherein the multi-mode valve is coupled to the auxiliary chamber and the auxiliary lumen is coupled to at least one of:
a suction source; and
a pressure source.

11. The ET tube of claim 1, wherein at least a portion of said elongated tube has a cross-sectional shape that is one of:
substantially round;
substantially oval;
substantially egg-shaped; and
substantially triangular.

12. The ET tube of claim 1, wherein said cuff has a cross-sectional shape that is one of:
substantially round;
substantially oval;
substantially egg-shaped; and
substantially triangular.

13. The ET tube of claim 1, wherein the multi-mode valve comprises at least the following modes of operation:
a normally sealed or closed mode;
at least one open mode allowing fluid and/or gas to pass into and/or out of the elongated tube.

14. The ET tube of claim 1, wherein the multi-mode valve comprises the following modes of operation:
a normally sealed or closed mode;
a first open mode allowing fluid and/or gas to pass into the elongated tube; and
a second open mode allowing fluid and/or gas to pass out of the elongated tube.

15. The ET tube of claim 1, wherein the multi-mode valve comprises the following modes of operation:
a normally sealed or closed mode;
a first open mode allowing fluid and/or gas to pass into the auxiliary lumen; and
a second open mode allowing fluid and/or gas to exit out of the multi-mode valve from the auxiliary lumen.

16. The ET tube of claim 1, wherein the multi-mode valve is a three-way valve.

17. The ET tube of claim 1, wherein the multi-mode valve comprises at least one of:
a slit valve;
at least two deflectable edges arranged adjacent one another;
a valve positioned arranged an outer wall of the elongated tube and being substantially flush with an outer surface of the outer wall of the elongated tube;
a valve integrally formed in an outer wall of the elongated tube and being substantially flush with an outer surface of the outer wall of the elongated tube;
a fluid-flow regulating device;
a passive fluid-flow regulating device; and
a pressure responsive fluid-flow regulating device.

18-28. (canceled)

29. A method for tracheal intubation, comprising:
inserting the ET tube of claim 1 into a trachea; and
inflating the inflatable cuff to enable positive pressure ventilation.

30. (canceled)

31. An endotracheal (ET) tube, comprising:
an elongated tube having a main lumen, an auxiliary chamber, a proximal end, and a distal end;
an inflatable cuff arranged on said elongated tube between said proximal and distal ends; and
a fluid-flow regulating device arranged on the elongated tube and in fluid communication with the auxiliary chamber,
wherein the fluid-flow regulating device comprises the following modes of operation:
a normally sealed or closed mode;
a first open mode allowing fluid and/or gas to pass into the elongated tube; and
a second open mode allowing fluid and/or gas to pass out of the elongated tube.

32. An endotracheal (ET) tube, comprising:
an elongated tube having a main lumen, an auxiliary chamber, a proximal end, and a distal end;
an inflatable cuff arranged on said elongated tube between said proximal and distal ends; and
a multi-mode valve integrally formed with an outer wall of the elongated tube and in fluid communication with the auxiliary chamber,
wherein the multi-mode valve comprises the following modes of operation:
a normally sealed or closed mode;
a first open mode allowing fluid and/or gas to pass into the auxiliary chamber based on a first pressure difference between opposite sides of the multi-mode valve; and
a second open mode allowing fluid and/or gas to pass out of the auxiliary chamber based on a second pressure difference between opposite sides of the multi-mode valve.
